(19) United States
(12) Patent Application Publication
(10) Pub. No.: US 2006/0258601 A1
(43) Pub. Date: Nov. 16, 2006

(54) Formulation for inwardly transferring nucleic acids into eucaryotic cells

(76) Inventors: Markus Hecker, Gottingen (DE); Andreas H. Wagner, Göttingen (DE)

Correspondence Address: FULBRIGHT & JAWORSKI L.L.P., 600 CONGRESS AVE., SUITE 2400, AUSTIN, TX, 78701, US

(21) Appl. No.: 10/526,430

(22) PCT Filed: Sep. 2, 2003
(86) PCT No.: PCT/DE03/02901

§ 371 Date: Feb. 13, 2006

(30) Foreign Application Priority Data
Sep. 2, 2002 (DE) 102 40 418.6

(51) Int. Cl. A61K 48/00; A61K 31/192

(52) U.S. Cl. 514/44; 514/570

(57) ABSTRACT

The present invention relates to a pharmaceutical formulation for the funnelling of nucleic acids into eukaryotic cells, characterised in that the formulation has a pH-value within the range of pH 6.0 to pH 7.4, and/or an anion concentration within the range from 5 to 100 mmol/l and/or provides nonsteroidal anti-inflammatory drugs with a concentration within the range from 10 to 500 μmol/l.

The present invention relates to a pharmaceutical formulation for funnelling nucleic acids into eukaryotic cells, characterised in that the formulation has a pH value within the range from pH 6.0 to pH 7.4, and/or an anion concentration within the range from 5 to 100 mmol/l and/or nonsteroidal anti-inflammatory drugs with a concentration within the range from 10 to 500 μmol/l.

One substantial goal of deciphering the human genome is to identify pathogenic genes (on the basis of the mode of action of their products) and/or to identify pathogenic changes in the structure of these genes (polymorphisms) and to allocate them to a disease profile. If it is accepted that such diseases are caused by a defined number of gene products expressed too strongly, too weakly or incorrectly, this research will bring closer the causal treatment of a plurality of diseases. In fact, for a whole series of inherited diseases (e.g. mucoviscidosis), the generally single genetic defect (monogenetic disease) is already known; however, the situation is considerably more complex for other disorders (e.g. high blood pressure). These diseases are evidently not the result of a single genetic defect but rather of multiple genetic defects (polygenetic disease), which predestine the affected persons to develop the disease on exposure to certain environmental factors. Regardless of this limitation, the targeted intervention into the expression of one or more genes does offer the opportunity for a cause-related and not merely symptom-related therapy.

According to the current state of scientific knowledge, four options are available for such “gene therapy”. For instance, it is now readily possible to funnel a substitute gene into body cells using a gene-carrier and to have it transcribed by the cell's own protein-synthesis mechanisms into the corresponding protein (liposomal transfer of a plasmid, transient expression) and/or to integrate this gene into the genome of the target cells (viral gene transfer, stable expression). However, major difficulties are still encountered in the correct addressing of the target cells, in transfer efficiency and where required, in the switching on and switching off of the transferred gene. Moreover, the liposomal and viral transfer systems currently used often have a cell-damaging effect or trigger a potentially dramatic, immunologically-determined intolerance reaction.

In order to prevent the expression of a pathogenic gene, by way of contrast with the gene-transfer technique, this can, for the first time, be blocked specifically during the so-called translation of the messenger-RNA (mRNA) into the corresponding protein. With this antisense technique, short, single DNA strands (generally comprising 15-25 nucleotides) are funnelled into the target cells, which provide a base sequence complementary for their target-mRNA. Depositing the antisense oligonucleotides on the similarly single-strand mRNA (DNA-RNA-hybridisation) leads to an interruption of the translation. By contrast, with the second of option of this kind, so-called RNA-interference (RNAi), an RNA-double strand comprising exactly 21 base pairs is funnelled into the cell, of which the sequence is identical to a segment of the mRNA coding for the target protein. Following this, a complex of proteins, which is not yet known in detail, is formed in the target cell; this specifically splits the target mRNA and accordingly prevents its translation. Both techniques share one problem: the single DNA strands and the double RNA strands respectively appear not to be absorbed into the target cells of their own accord, but must, like the considerably larger plasmids (generally several thousand base pairs long), be transfected into them. For this purpose, they are generally packaged in liposomes, which act as a transport medium.

The third method for targeted intervention into the gene expression uses short DNA double strands, so-called decoy oligonucleotides. The first stage in the expression of a gene is the transcription of the corresponding DNA segment on the chromosome into an RNA single strand. So-called transcription factors are critical for the initiation of the transcription. These regulatory proteins bind to the starter region of the gene (promoter region) and initiate the transcription of the gene through RNA polymerase. Transcription factors bound to the DNA can also block this transcription process. Decoy oligonucleotides are short DNA double strands (generally comprising 15-25 base pairs), which imitate the sequence motif, to which the target transcription factor binds in the starter region of its (their) target gene (target genes). Every transcription factor recognises only its corresponding sequence motif; in this manner, the decoy oligonucleotide approach is specific.

The consequence of the transcription factor being neutralised as a result of the decoy oligonucleotides in the cytoplasm or in the cell nucleus is that this can no longer induce or block the expression of its (their) target gene (genes).

There is therefore an urgent need for a simple means of funnelling nucleic acids, which does not place the cells or the organism under stress.

This object is achieved by the subject matter defined in the claims.

The invention is explained in greater detail with reference to the following drawings.

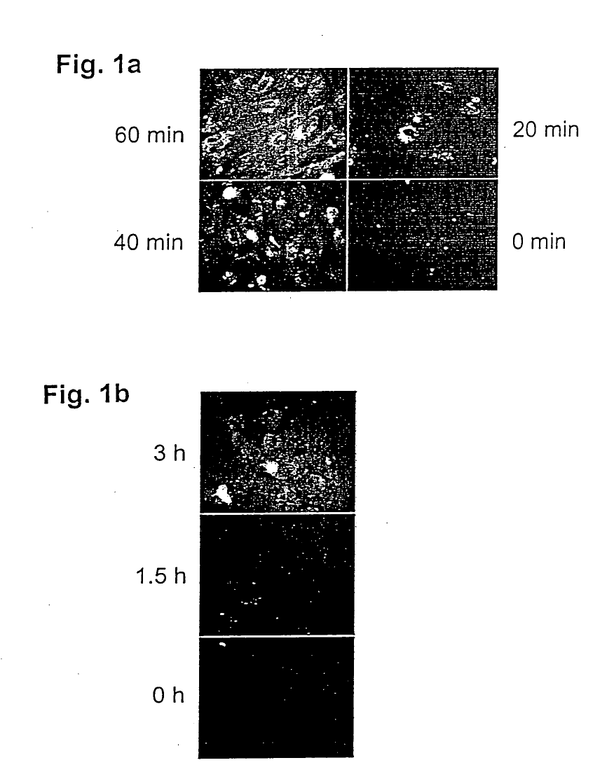

FIG. 1 shows, by way of example, the results of the time-dependent absorption of an FITC-marked decoy oligonucleotide against the transcription factor C/EBP (10 μmol/l; FIG. 1a) and an FITC-marked decoy oligonucleotide against the transcription factor AP-1 (10 μmol/l; FIG. 1b) in human cultivated endothelial cells, which had been incubated in cell-culture medium. The absorption of fluorescence-dye-marked nucleic acids was demonstrated by means of fluorescence microscopy (magnification 400×).

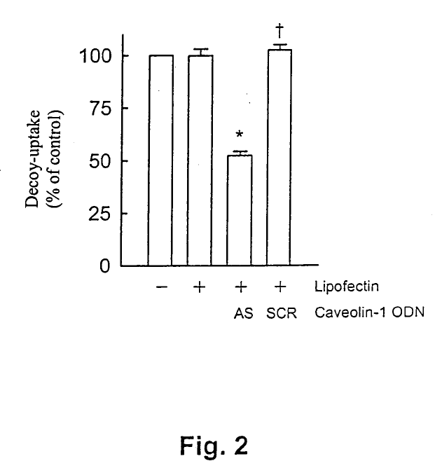

FIG. 2 shows, in the form of a bar chart, the effect of an antisense oligonucleotide (AS)-supported reduction of the protein expression of caveolin-1 (37±10% of the control, n=3) in human cultivated endothelial cells on the absorption of the FITC-marked C/EBP decoy oligonucleotide (10 μmol/l) over a period of 1 hour. Statistical summary (n=3-4, related as a percentage to the absorption of the decoy oligonucleotide in un-treated control cells; *P<0.05 versus control, †P<0.05 versus AS). SCR (scrambled) indicates the treatment of the endothelial cells with an oligonucleotide of the same base composition but different sequence from the antisense oligonucleotide.

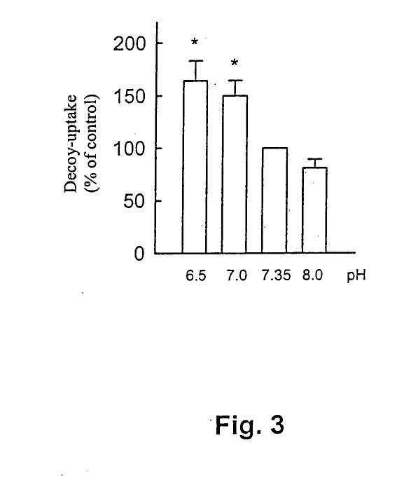

FIG. 3 shows, in the form of a bar chart, the effect of a change of the extracellular pH-value on the absorption of the FITC-marked C/EBP decoy oligonucleotide (10 μmol/l) in human cultivated endothelial cells over a period of 1 hour. Statistical summary (n=4-5, related as a percentage to the absorption of the decoy oligonucleotide at pH value 7.35; *P<0.05).

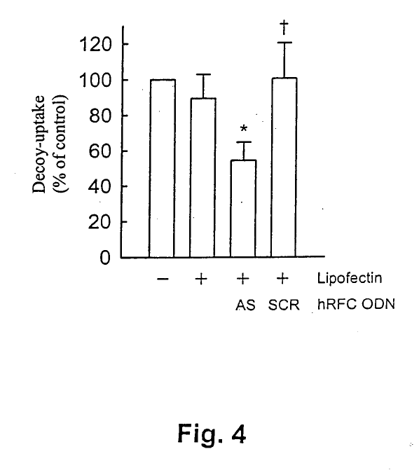

FIG. 4 shows, in the form of a bar chart, the effect of an antisense oligonucleotide (AS)-supported reduction of the protein expression of the reduced folic-acid-carrier (on 33±10% of the control, n=5) in human cultivated endothelial cells on the absorption of the FITC-marked C/EBP-decoy oligonucleotide (10 μmol/l) over a period of 1 hour. Statistical summary (n=3, related as a percentage to the absorption of the decoy oligonucleotide in un-treated control cells; *P<0.05 versus control, †P<0.05 versus AS). SCR (scrambled) indicates the treatment of the endothelial cells with an inactive antisense oligonucleotide.

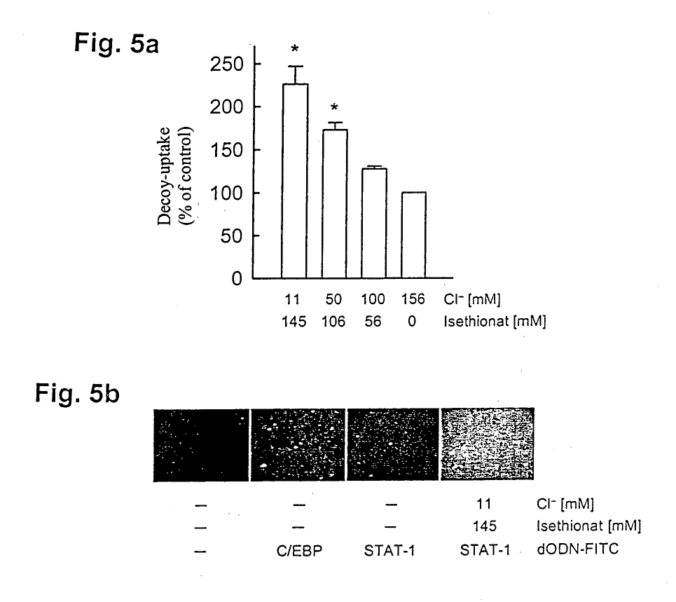

FIG. 5 shows, in the form of a bar chart (a), the effect of a change of the extracellular chloride-ion concentration (gradual substitution by isethionate) on the absorption of the FITC-marked C/EBP-decoy oligonucleotide (10 μmol/l) in human cultivated endothelial cells over a period of 1 hour. Statistical summary (n=4, related as a percentage to the absorption of the decoy oligonucleotide with 156 mmol/l Cl⁻; *P<0.05). FIG. 5(b) shows, by way of example, the effect of a reduction of the extracellular chloride concentration from 156 to 11 mmol/l on the absorption of an FITC-marked STAT-1-decoy oligonucleotide in human cultivated endothelial cells over a period of 1 hour (fluorescence microscopy images, magnification 200×).

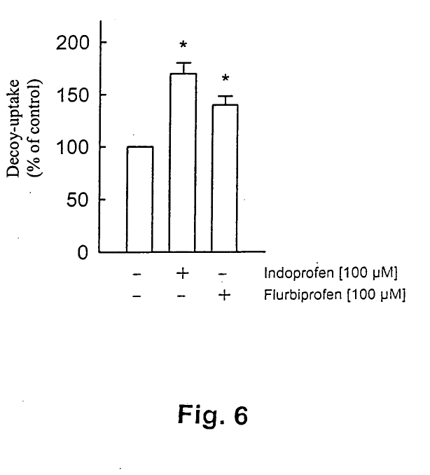

FIG. 6 shows, in the form of a bar chart, the effect of a co-incubation with flurbiprofen or indoprofen (in each case 100 μmol/l) on the absorption of the FITC-marked C/EBP-decoy oligonucleotide (10 μmol/l) in human cultivated endothelial cells over a period of 1 hour. Statistical summary (n=4, related as a percentage to the absorption of the decoy oligonucleotides in un-treated control cells; *P<0.05).

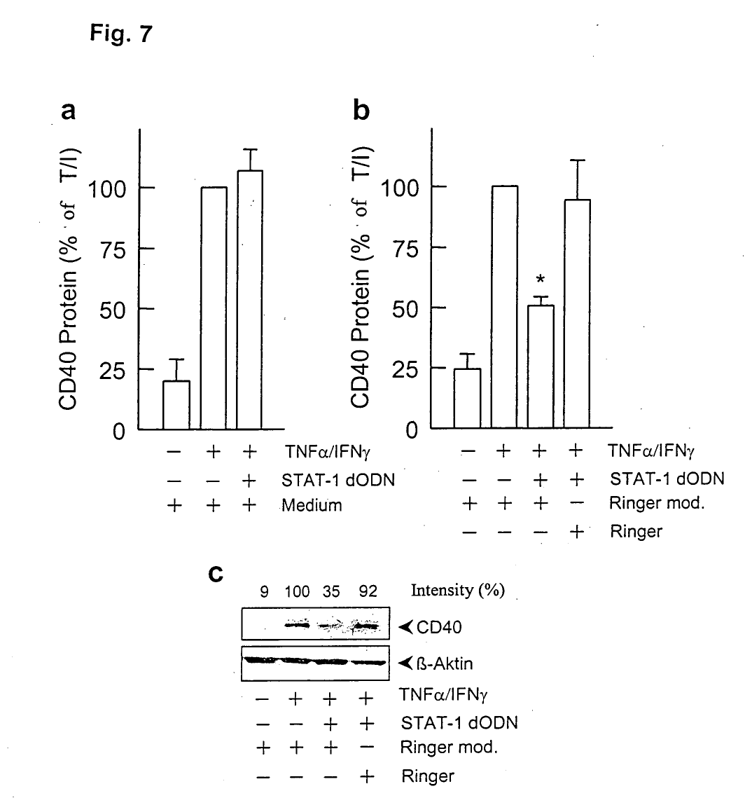

FIG. 7 shows, in the form of bar charts (a,b) and in a representative Western-blot analysis (c), the effect of (a) cell culture medium (n=3) and (b,c) un-modified and respectively modified (mod) Ringer's solution (11 mmol/l chloride ions, pH 7.0) as incubation medium on the STAT-1 decoy oligonucleotide-mediated inhibition of cytokine-stimulated (100 U/ml tumour necrosis factor a [TNFa] plus 1000 U/ml interferon-γ [IFNγ] for 10 hours) CD40 protein expression in human cultivated endothelial cells (related as a percentage to the quantity of protein in cytokine-stimulated cells [T/I]) *P<0.05 versus T/I; b, statistical summary, n=6; c, representative Western-blot analysis with β-actin as internal standard. The endothelial cells were pre-incubated with un-marked decoy oligonucleotides (10 μmol/l) for 30 minutes before exposure to the cytokines.

By contrast with plasmids, antisense and RNAi oligonucleotides, decoy oligonucleotides (double-strand DNA oligonucleotides) can evidently enter the relevant target cell without auxiliary agents (transfection agents). The mechanism underlying this transport was hitherto unknown. The inventors have now succeeded in explaining this mechanism. On the basis of the knowledge obtained in this context, new formulations are provided for the introduction of nucleic acids into eukaryotic cells, especially mammalian cells and, in particular human cells.

The term “formulation” or “pharmaceutical formulation” as used in the present document means the pharmaceutical form of preparation, for example, for a drug or an inoculation medium, which is administered in vivo to a human or an animal, or in vitro or ex vivo to organs, tissue or cells, consisting of one or more active ingredients and auxiliary formulation agents. Active ingredients according to the present invention are nucleic acids.

The term “auxiliary formulation agents” as used in present document means all ingredients of the pharmaceutical preparation mentioned above with the exception of the active ingredients. Auxiliary formulation agents can be, for example, physiological salt or buffer solutions, water, preserving agents, ions, acids, bases, preserving solutions for organ transplantation, blood replacement fluids, inhalation, infusion and injection solutions and medicines.

The present invention relates to a new formulation for the funnelling of nucleic acids into eukaryotic cells, characterised in that the formulation has a pH-value within the range from pH 6.0 to pH 7.4, preferably within the range from approximately pH 6.2 to approximately pH 7.0 and by particular preference of approximately pH 6.5 or pH 7.0, and/or an anion concentration, preferably a chloride-ion concentration within the range from approximately 5 to approximately 100 mmol/l, preferably within the range from approximately 5 to approximately 50 mmol/l and by particular preference within the range from approximately 5 to approximately 10 mmol/l, and/or nonsteroidal anti-inflammatory drugs, e.g. flurbiprofen or indoprofen, with a concentration within the range from approximately 10 to approximately 500 μmol/l, preferably within the range from approximately 50 to approximately 250 μmol/l and by particular preference with a concentration of approximately 100 μmol/l. Moreover, in addition to the active ingredients and the features described above, the formulation may also contain one or more suitable buffers. An example of a suitable buffer is a modified Ringer's solution containing 145 mmol/l Na⁺, 5 mmol/l K⁺, 11 mmol/l Cl⁻, 2 mmol/l Ca²⁺, 1 mmol/l Mg²⁺, 10 mmol/l Hepes, 145 mmol/l isethionate, 10 mmol/l D-glucose, wherein the pH-value is within the range from 6.5 to 7.0, preferably approximately 6.5 or 7.0.

Initially, the inventors observed that the intracellular distribution of the fluorescence-dye-marked decoy oligonucleotides absorbed by the human endothelial cells investigated is heterogeneous. Alongside accumulations in vesicle-like structures, a more strongly diffuse marking of cytoplasm and cell nucleus was shown. Especially the accumulation of nucleic acids in vesicles gave grounds for the assumption that the absorption process could be a receptor-mediated, endocytosis-like process.

It was subsequently shown, that, like smooth vascular muscle cells or monocytes, human endothelial cells express one or both variants of the folic-acid receptor, a potential candidate for the absorption of nucleic acids in the cells. This receptor is preferably localised in so-called caveolae in the cell membrane. The destruction of the caveolae—through the withdrawal of cholesterol or the inhibition of the expression of caveolin-1 (FIG. 2)—led to a significant restricttion of the absorption of the decoy oligonucleotide.

By contrast, lowering the extracellular pH-value favours the receptor-mediated folic-acid binding (affinity), and this pH-dependence was also shown for the absorption of the decoy oligonucleotides into the human endothelial cells (FIG. 3). After the binding of the folic acid to the receptor, the caveolae are internalised (potocytosis; R G W Anderson (1998) Annu. Rev. Biochem, 67, 199). In order to release the folic acid enclosed in these vesicles into the cytoplasm, an anion transporter (carrier) is required, which can be inhibited by probenicid (Kamen et al. (1991) J. Clin. Invest. 87, 1442) and which is not identical to the reduced folic-acid carrier hFRC described below. In fact, the accumulation of decoy oligonucleotides in the human endothelial cells was also probenicid-sensitive.

The formulation according to the invention therefore relates to a formulation with a pH-value within the range from approximately pH 6.0 to approximately pH 7.4. The pH value is preferably within the range from pH 6.2 to approximately pH 7.0 and by particular preference approximately pH 6.5 or 7.0.

Alongside receptor-mediated potocytosis, the primary transport route for folic acid into mammalian cells is absorption via the reduced-folic acid carrier hRFC (L H Matherly (2001) Prog. Nucleic Acid Res. Mol. Biol. 67, 131). In principle, this transporter should be available to every body cell which is capable of cell division, because folic acid is essential for DNA synthesis (see also Whetstine et al. (2002) Biochem. J. July 29 (epub ahead of print]). Human endothelial cells also express hFRC. The antisense oligonucleotide-supported reduction of the expression of the hFRC protein to one-third of these cells led to an inhibition of the decoy-oligonucleotide absorption by 45% (FIG. 4). Further indications regarding the participation of this transport system in decoy-oligonucleotide absorption (see characteristics of hFRC described in L H Matherly (2001) Prog. Nucleic Acid Res. Mol. Biol. 67, 131) were their considerably improved inhibition by the antifolate methotrexate by comparison with folic acid and the high sensitivity to the anionic-exchange inhibitor DIDS (4,4′-diisothiocyano-2,2′-stilben-disulfonic acid).

In fact, for the hFRC-mediated absorption of the anionic folic acid into mammalian cells, it is necessary, as a counter-move, for an anion, preferably chloride, to leave the cell (antiport) and/or for a cation, preferably a proton (H⁺) to be co-transported into the cell (symport) However, since the carrier has the maximum affinity for folic acid and/or methotrexate at a quasi physiological pH-value of 7.5, a lowering of the extracellular pH (that is, a rise in the proton concentration) fails to achieve the desired effect of improving nucleic acid absorption via this transport route. Facilitating the chloride transport out of the cell is more promising, e.g. by reducing the extracellular chloride concentration (typically 120 mmol/l), preferably below the intracellular value (12 mmol/l), thereby creating an outwardly-directed concentration gradient for chloride. As shown in FIG. 5, the reduction of the extracellular chloride concentration did in fact lead to a significant improvement in the decoy-oligonucleotide absorption into human endothelial cells.

In summary, the findings reported above confirm that, alongside the pH-sensitive folate-receptor-mediated potocytosis, the absorption of nucleic acids into human cells takes place via the reduced folic acid carrier, and the efficiency of this transport route can be considerably increased by lowering the extracellular anion concentration, especially the chloride concentration.

The formulation according to the invention therefore relates to a formulation comprising an anion concentration, preferably a chloride-ion concentration within the range from approximately 5 to approximately 100 mmol/l, preferably within the range from approximately 5 to approximately 50 mmol/l and by particular preference within the range from approximately 5 to approximately 10 mmol/l. Furthermore, the physiological substitution of chloride ions can be achieved, for example, by the addition of an equimolar quantity of isethionate.

Alongside the absorption of a substance, its expulsion also plays an important role for its momentary concentration and/or availability in the cell. Such a transport route for folic acid out of mammalian cells, which can be inhibited by anti-inflammatory drugs (nonsteroidal anti-inflammatory drugs), such as flurbiprofen or indoprofen, has been described (M Saxena, G B Henderson (1996) Biochem. Pharmacol. 51, 974). As shown in FIG. 6, decoy oligonucleotides are also removed from human cells via this transport route; that is to say, the concentration of the nucleic acids in the cell can be significantly increased by a blockade of this transport route.

The formulation according to the invention therefore also relates to a formulation comprising nonsteroidal anti-inflammatory drugs such as flurbiprofen or indoprofen in a concentration within the range from approximately 10 to approximately 500 μmol/l, preferably within the range from approximately 50 to approximately 250 μmol/l and by particular preference in a concentration of approximately 100 μmol/l.

The transport routes described above can also be used by other nucleic acids, e.g. by single-strand RNA/DNA oligonucleotides or by double-strand RNA oligonucleotides, to a comparable extent and in addition to decoy oligonucleotides, and are not restricted to endothelial cells. For example, FITC-marked single-strand DNA oligonucleotides were transported as effectively into human endothelial cells as the corresponding double-strand (decoy) oligonucleotides, and the rate of absorption of decoy oligonucleotides into human endothelial and smooth vascular muscle cells was generally identical.

Apart from the condition in principle that, for example, decoy oligonucleotides effectively neutralise their target transcription factor, it is critical for the therapeutic efficacy of nucleic acids that they are absorbed rapidly and to an adequate extent into the target cell without the need for potentially cytotoxic auxiliary agents. To this extent, preferred methods of the present invention for the application of these nucleic acids comprise the use of appropriate buffers with:
1. A pH value within the range from approximately pH 6.0 to pH 7.4, preferably within the range from approximately pH 6.2 to pH 7.0 and by particular preference approximately pH 6.5 or 7.0 and/or
2. An extracellular anion concentration, preferably a chloride concentration (e.g. through the addition of isethionate) within the range from approximately 5 to approximately 100 mmol/l, preferably within the range from approximately 5 to approximately 50 mmol/l and by particular preference within the range from approximately 5 to approximately 10 mmol/l, and/or
3. Nonsteroidal anti-inflammatory drugs, preferably flurbiprofen or indoprofen, with a concentration in the range from approximately 10 to approximately 500 μmol/l, preferably within the range from approximately 50 to approximately 250 μmol/l and by particular preference with a concentration of approximately 100 μmol/l.

Moreover, the present invention relates to a formulation for funnelling nucleic acids into eukaryotic cells, in which two or all of the above-named features can be combined. FIG. 7 shows an example of the increased biological activity of the nucleic acids achieved as a result. One preferred formulation comprises a combination of the adjustment of pH-value and chloride-concentration according to the invention.

In one preferred embodiment, a formulation according to the invention, which is brought into contact with the target cells, contains only nucleic acids (in a concentration from 0.01 to 100 μmol/l) and a buffer. One or more appropriate buffers can be used. An example of a buffer of this kind is a modified Ringer's solution containing 145 mmol/l Na⁺, 5 mmol/l K⁺, 11 mmol/l Cl⁻, 2 mmol/l Ca²⁺, 1 mmol/l Mg²⁺, 10 mmol/l Hepes, 145 mmol/l isethionate, 10 mmol/l D-glucose, pH 6.5 or pH 7.0, as used in the experiment shown in FIG. 7.

The formulation used in the method according to the present invention is preferably applied locally by injection, infusion, inhalation, or any other form of application, which allows local access. The ex vivo application of the formulation (incubation of blood vessels, tissue or cells), used within the method of the present invention, also allows a local access. The goal is to bring the nucleic acid-containing mixture as close as possible to the cells to be treated and—at least for a short time—to create an optimum extracellular environment for the absorption of the nucleic acids into the target cells.

The following examples are provided merely by way of explanation and in no sense restrict the scope of invention.

1. Cell Culture

Human endothelial cells were isolated from umbilical veins by treatment with 1.6 U/ml dispase in Hepes-modified tyrode solution for 30 minutes at 37° C. and cultivated on gelatine-coated 6-well tissue-culture dishes (2 mg/ml gelatine in 0.1 M HCl for 30 minutes at room temperature) in 1.5 ml M199 medium (Gibco Life Technologies, Karlsruhe, Germany), containing 20% foetal calf serum, 50 U/ml penicillin, 50 μg/ml streptomycin, 10 U/ml nystatin, 5 mM HEPES and 5 mM TES, 1 μg/ml heparin and 40 μg/ml endothelial growth factor. The cells were identified by their typical pavement morphology, positive immune-staining for von Willebrandt-Factor (vWF) and fluorimetric demonstration (FACS) of PECAM-1 (CD31) and negative immuno-staining for smooth muscular α-actin (Krzesz et al. (1999) FEBS Lett. 453, 191).

Human smooth vascular muscle cells were isolated from the veins of excised thymus glands. After the removal of adhering connective issue and fatty tissue, the blood vessel was mechanically comminuted using a scalpel. Following this, the tissue was incubated at 37° C. and with 5% CO₂ for 14-16 hours in a digestive solution (5% foetal bovine serum, 5 mmol/l HEPES, 5 mmol/l TES, 50 U/ml penicillin, 50 μg/ml streptomycin, 10 U/ml nystatin and 0.15% collagenase (Clostridium histolyticum, Sigma-Aldrich, Deisenhofen) in DMEM medium; Gibco Life Technologies). After centrifuging of the cell suspension at 1000 rpm for 5 minutes at room temperature, the cell pellet was suspended in 2-3 ml growth medium (Smooth Muscle Cell Growth Medium 2, PomoCell GmbH, Heidelberg) and flattened out into tissue culture dishes, which had previously been coated with gelatine (2 mg gelatine per ml 0.1 N HCl) for at least 30 minutes at room temperature and then washed twice with the medium. The growth medium was replaced under sterile conditions after 2 days and the cells were briefly washed with medium. In the subsequent period, the medium was changed every 4 days.

The human monocyte cell line THP-1 (ATCC TIB 202) was cultivated in RPMI 1640 medium (Gibco Life Technologies), containing 10% foetal calf serum, 50 U/ml penicillin, 50 μg/ml streptomycin and 10 U/ml nystatin.

2. Decoy Oligonucleotide Synthesis

Double-strand decoy oligonucleotides were manufactured from the complementary single-strand, fluorescein isothiocyanate (FITC)-marked oligonucleotides (Eurogentec, Köln, Germany) as described in Krzesz et al. (1999) FEBS Lett. 453, 191. The single-strand sequences of the oligonucleotides were as follows (underlined letters indicate phosphorothioate-coupled bases):

(SEQ ID NO: 1)

| | |
|---|---|
| AP-1, | 5′-CGCTTGATGACTCAGCCGGAA-3′ |

(SEQ ID NO: 2)

| | |
|---|---|
| C/EBP, | 5′-TGCAGATTGCGCAATCTGCA-3′ |

(SEQ ID NO: 3)

| | |
|---|---|
| STAT-1, | 5′-CATGTTATGCATATTCCTGTAAGTG-3′ |

3. Antisense Oligonucleotide Synthesis and Incubation

For an antisense mixture, 3% lipofectin (v/v) (Gibco Life Technologies) was added to 1 ml culture medium and incubated for 60 minutes at room temperature (RT). Following this, the corresponding antisense or control oligonucleotide (Eurogentec, Köln, Germany) was added in a final concentration of 0.5 μmol/l and incubated for a further 30 minutes at room temperature. At the start of the experiments, the corresponding quantities of heparin and endothelial growth factor were added, and the conventional cell culture medium of the endothelial cell culture was replaced by the antisense lipofectin medium. After 6 hours, the antisense lipofectin medium was removed and replaced by fresh cell culture medium; the Western-blot analysis and/or the fluorescence-microscopic analysis of the decoy-oligonucleotide absorption was carried out 24 hours after the transfection.

The sequence (phosphorothioester bonds are marked *) of the antisense oligonucleotide for caveolin-1 was 5′-A*T*G*TCCCTCCGAGT*C*T*A-3′ (SEQ ID NO:4); as a control, a scrambled oligonucleotide with identical base composition to the antisense oligonucleotide but with a different sequence (5′-C*T*C*GATCCTGACTA*C*T*G-3′) (SEQ ID NO:5) was used. The sequence of the antisense oligonucleotide for the reduced folate carrier (hRFC) was 5′-C*A*A*A*GG*T*A*GC*A*C*A*CG*A*G-3′ (SEQ ID NO:6). Here also, a scrambled oligonucleotide was used as the control (5′-A*C*A*T*GG*A*C*A*CG*A*A*GC*A*G-3′) (SEQ ID NO:7).

4. RT-PCR Analysis

The total cellular RNA was isolated using the Qiagen RNeasy Kit (Qiagen, Hilden, Germany); following this, a cDNA-synthesis was implemented with a maximum of 3 μg RNA and 200 U Superscript™ II Reverse Transcriptase (Gibco Life Technologies) in a total volume of 20 μl in accordance with the manufacturer's instructions. For the subsequent polymerase chain reaction, 5 μl of the cDNA- and 1 U Taq DNA polymerase (Gibco Life Technologies) were used in a total volume of 50 μl. The PCR products were separated on 1.5% agarose gels containing 0.1% ethidium bromide, and the intensity of the bands was measured densiometrically with a CCD camera system and recorded with the One-Dscan gel analysis software manufactured by Scanalytics (Billerica, Mass., USA).

All of the PCR reactions were carried out individually for each primer pair in a Tpersonal Cycler (Biometra, Göttingen, Germany):

hFR1 (folate receptor α), product size 181 bp, 37 cycles, addition temperature 60° C., (forward primer), 5′-CAAGGTCAGCAACTACAGCCGAGGG-3′ (SEQ ID NO:8), (reverse primer) 5′-TGAGCAGCCACAGCAGCATTAGGG-3′ (SEQ ID NO:9).

hFR2 (folate receptor b), product size 385 bp, 37 cycles, addition temperature 61° C., (forward primer), 5′-CTGTGTAGCCACCATGTGCAGTGC-3′ (SEQ ID NO;10), (reverse primer) 5′-TGTGACAATCCTCCCACCAGCG-3′) (SEQ ID NO:11).

h1FRC, product size 333 bp, 37 cycles, addition temperature 60° C., (forward primer), 5′-CCAAGCGCAGCCTCTTCTTCTTCAACC-3′ (SEQ ID NO:12), (reverse primer) 5′-CCAGCAGCTGGAGGCAGCATCTGCC-3′ (SEQ ID NO:13); Sprecher et al., (1998) Arch. Dermatol. Res. 290, 656).

h2FRC2, product size 167 bp, 37 cycles, addition temperature 56° C., (forward primer), 5′-CCATCGCCACCTTTCAGATTGC-3′ (SEQ ID NO:14), reverse primer 5′-CGGAGTATAACTGGAACTGCTTGCG-3′ (SEQ ID NO:15).

The identity of all PCR products was confirmed by subsequent sequencing.

5. Western-Blot Analysis

The human umbilical vein endothelial cells were opened by freezing successively five times in liquid nitrogen and thawing at 37° C. Protein extracts were manufactured as described by Hecker et al. (1994) Biochem J. 299, 247. 20-30 μg protein were separated using a 10% polyacrylamide gel electrophoresis under denaturing conditions in the presence of SDS according to a standard protocol and transferred to a BioTrace™ polyvinylidene fluoride transfer membrane (Pall Corporation, Rossdorf, Germany). A polyclonal primary anti-human antibody from BD Biosciences, Heidelberg Germany was used for the immunological demonstration of caveolin-1. A polyclonal anti-human antibody (generously provided by Dr. Hamid M. Said, Veterans Affairs Medical Center, Long Beach, Calif. USA) was used for the demonstration of the hFRC protein. CD40 protein was detected with a polyclonal anti-human antibody (Research Diagnostics Inc., Flanders, N.J., USA). The protein bands were visualised after the addition of a peroxidase-coupled anti-mouse IgG and/or anti-rabbit IgG (1:3000, Sigma, Deisenhofen, Germany) using the chemi-luminescence method (SuperSignal Chemiluminescent Substrate; Pierce Chemical, Rockford, Ill., USA) and subsequent autoradiography (Hyperfilm™ MP, Amersham Pharmacia Biotech, Buckinghamshire, England). The application and transfer of identical protein quantities was shown, after “stripping” the transfer membrane (5 minutes 0.2 N NaOH, followed by 3×10 minutes washing with H₂O), by the demonstration of identical protein bands of β-actin with a monoclonal primary antibody and a peroxidase-coupled anti-mouse IgG (both by Sigma-Aldrich, 1:3000 dilution).

6. Fluorescence Microscopy

Before the start of the experiment, the endothelial cells cultivated in the 24-well cell-culture plates were washed once with Ringer's solution at 37° C. (composition: 145 mmol/l Na⁺, 5 mmol/l K⁺, 156 mmol/l Cl⁻, 2 mmol/l Ca²⁺, 1 mmol/l Mg²⁺, 10 mmol/l Hepes, 10 mmol/l D-glucose, pH 7.35). Following this, 150 μl modified or respectively non-modified Ringer's solution were applied, depending on the experimental mixture, to the cells at 37° C., and the FITC-marked decoy oligonucleotide was added in a final concentration of 10 μmol/l. After an incubation period of up to 180 min at 37° C. and in ambient air, the cells were washed three times with 1 ml warm, non-modified Ringer's solution. The fluorescence intensities were recorded with the MicroMax CCD-camera (Princeton Instruments Inc., Trenton, N.J., USA), which was coupled to an Axiovert S100 TV microscope (Zeiss, Göttingen, Germany), with an excitation wavelength of 494 nm, an emission wavelength of 518 nm and 200× magnification. The fluorescence images (one image was taken for each portion of the experimental mixture) and the subsequent quantification was implemented using the MetaMorph V3.0 Software (Universal Imaging West Chester, Pa., USA). For the quantification, all fluorescence images for an experimental mixture were initially calibrated to an identical level of brightness and contrast. Following this, the software was used to determine an overall brightness integrated across the individual pixels for each image as a measure for the fluorescence intensity, thereby representing the intracellular concentration of the decoy oligonucleotide.

7. Statistical Analysis

Unless otherwise indicated, all data in the diagrams are shown as a mean value±SEM of n experiments. The statistical evaluation was implemented by one-sided variance analysis (ANOVA) followed by a Dunnett Post Test. A P-value of <0.05 was taken as a statistically significant difference.

1-10. (canceled)

11. A pharmaceutical formulation comprising a nucleic acid, wherein said formulation comprises a pH-value within the range from pH 6.2 to pH 7.0, and/or a chloride ion concentration within the range from 5 to 100 mmol/l and/or provides a nonsteroidal anti-inflammatory drug with a concentration within the range from 10 to 500 μmol/l.

12. The formulation according to claim 11, wherein the pH value is 6.5 or 7.0.

13. The formulation according to claim 11, wherein the chloride ions have a concentration within the range from 5 to 50 mmol/l.

14. The formulation of claim 11, wherein the chloride ions have a concentration within the range from 5 to 10 mmol/l.

15. The formulation of claim 11, wherein the nonsteroidal anti-inflammatory drug has a concentration within the range from 50 to 250 μmol/l.

16. The formulation of claim 11, wherein the nonsteroidal anti-inflammatory drug has a concentration of 100 μmol/l.

17. The formulation claim 11, wherein the nonsteroidal anti-inflammatory drug is flurbiprofen or indoprofen.

18. The formulation according to claim 1, further comprising a carrier substance or additive.


SEQUENCE LISTING (63 sequences)

SEQ ID NO: 1 - DNA, 22 bp, Artificial Sequence
  Decoy-Oligonucleotide
agctcttccctggccggctgac

SEQ ID NO: 2 - DNA, 22 bp, Artificial Sequence
  Decoy-Oligonucleotide
gtcagccggccagggaagagct

SEQ ID NO: 3 - DNA, 22 bp, Artificial Sequence
  Decoy-Oligonucleotide
agctcttccctggctggctgac

SEQ ID NO: 4 - DNA, 22 bp, Artificial Sequence
  Decoy-Oligonucleotide
gtcagccagccagggaagagct

SEQ ID NO: 5 - DNA, 23 bp, Artificial Sequence
  Decoy-Oligonucleotide
cttccctggccggctgaccctgc

SEQ ID NO: 6 - DNA, 23 bp, Artificial Sequence
  Decoy-Oligonucleotide
gcagggtcagccggccagggaag

SEQ ID NO: 7 - DNA, 23 bp, Artificial Sequence
  Decoy-Oligonucleotide
cttccctggctggctgaccctgc

SEQ ID NO: 8 - DNA, 23 bp, Artificial Sequence
  Decoy-Oligonucleotide
gcagggtcagccagccagggaag

SEQ ID NO: 9 - DNA, 19 bp, Artificial Sequence
  Decoy-Oligonucleotide
gctcttccctggccggctg

SEQ ID NO: 10 - DNA, 19 bp, Artificial Sequence
  Decoy-Oligonucleotide
cagccggccagggaagagc

SEQ ID NO: 11 - DNA, 19 bp, Artificial Sequence
  Decoy-Oligonucleotide
caagctcttccctggccgg

SEQ ID NO: 12 - DNA, 19 bp, Artificial Sequence
  Decoy-Oligonucleotide
ccggccagggaagagcttg

SEQ ID NO: 13 - DNA, 19 bp, Artificial Sequence
  Decoy-Oligonucleotide
tcttccctggccggctgac

SEQ ID NO: 14 - DNA, 19 bp, Artificial Sequence
  Decoy-Oligonucleotide
gtcagccggccagggaaga

SEQ ID NO: 15 - DNA, 19 bp, Artificial Sequence
  Decoy-Oligonucleotide
ctggccggctgaccctgcc

SEQ ID NO: 16 - DNA, 19 bp, Artificial Sequence
  Decoy-Oligonucleotide
ggcagggtcagccggccag

SEQ ID NO: 17 - DNA, 16 bp, Artificial Sequence
  Decoy-Oligonucleotide
tccctggccggctgac

SEQ ID NO: 18 - DNA, 16 bp, Artificial Sequence
  Decoy-Oligonucleotide
gtcagccggccaggga

SEQ ID NO: 19 - DNA, 10 bp, Artificial Sequence
  Decoy-Oligonucleotide
ctggccggct

SEQ ID NO: 20 - DNA, 10 bp, Artificial Sequence
  Decoy-Oligonucleotide
agccggccag

SEQ ID NO: 21 - DNA, 10 bp, Artificial Sequence
  Decoy-Oligonucleotide
ctggctggct

SEQ ID NO: 22 - DNA, 10 bp, Artificial Sequence
  Decoy-Oligonucleotide
agccagccag

SEQ ID NO: 23 - DNA, 16 bp, Artificial Sequence
  Decoy-Oligonucleotide
tccctggcyggctgac

SEQ ID NO: 24 - DNA, 16 bp, Artificial Sequence
  Decoy-Oligonucleotide
gtcagccrgccaggga

SEQ ID NO: 25 - DNA, 13 bp, Artificial Sequence
  Decoy-Oligonucleotide
ctggcyggctgac

SEQ ID NO: 26 - DNA, 13 bp, Artificial Sequence
  Decoy-Oligonucleotide
gtcagccrgccag

SEQ ID NO: 27 - DNA, 16 bp, Artificial Sequence
  Decoy-Oligonucleotide
tccctbbcygbctgac

SEQ ID NO: 28 - DNA, 16 bp, Artificial Sequence
  Decoy-Oligonucleotide
gtcagvcrgvvaggga

SEQ ID NO: 29 - DNA, 13 bp, Artificial Sequence
  Decoy-Oligonucleotide
ccctbbcygbctg

SEQ ID NO: 30 - DNA, 13 bp, Artificial Sequence
  Decoy-Oligonucleotide
cagvcrgvvaggg

SEQ ID NO: 31 - DNA, 13 bp, Artificial Sequence
  Decoy-Oligonucleotide
ctbbcygbctgac

SEQ ID NO: 32 - DNA, 13 bp, Artificial Sequence
  Decoy-Oligonucleotide
gtcagvcrgvvag

SEQ ID NO: 33 - DNA, 10 bp, Artificial Sequence
  Decoy-Oligonucleotide
ctbbcygbct

SEQ ID NO: 34 - DNA, 10 bp, Artificial Sequence
  Decoy-Oligonucleotide
agvcrgvvag

SEQ ID NO: 35 - DNA, 21 bp, Artificial Sequence
  Decoy-Oligonucleotide
gagtctggccaacacaaatcc

SEQ ID NO: 36 - DNA, 21 bp, Artificial Sequence
  Decoy-Oligonucleotide
gacctctagggtcatgcaggt

SEQ ID NO: 37 - DNA, 19 bp, Artificial Sequence
  DNA Oligonucleotide
gggtcagccggccagggaa

SEQ ID NO: 38 - DNA, 21 bp, Artificial Sequence
  DNA Oligonucleotide
agcttgatgccctggtgggag

SEQ ID NO: 39 - DNA, 18 bp, Artificial Sequence
  Primer
ggaacctgtgtgaccctc

SEQ ID NO: 40 - DNA, 18 bp, Artificial Sequence
  Primer
ccacgtcatactcatcca

SEQ ID NO: 41 - DNA, 21 bp, Artificial Sequence
  Primer
gtactccacattcctacttct

SEQ ID NO: 42 - DNA, 22 bp, Artificial Sequence
  Primer
tttgggtctattccgttgtgtc

SEQ ID NO: 43 - DNA, 22 bp, Artificial Sequence
  Primer
ggacacccatcccaaatcagtc

SEQ ID NO: 44 - DNA, 22 bp, Artificial Sequence
  Primer
cacggtgaaatactgcctggtg

SEQ ID NO: 45 - DNA, 19 bp, Artificial Sequence
  Primer
tcaccatcttccaggagcg

SEQ ID NO: 46 - DNA, 20 bp, Artificial Sequence
  Primer
ctgcttcaccaccttcttga

SEQ ID NO: 47 - DNA, 20 bp, Artificial Sequence
  Primer
gttcatccggcaccagtcag

SEQ ID NO: 48 - DNA, 21 bp, Artificial Sequence
  Primer
acgtgcacatgagctgcctac

SEQ ID NO: 49 - DNA, 21 bp, Artificial Sequence
  Decoy-Oligonucleotide
cctgcattctgggaactgtag

SEQ ID NO: 50 - DNA, 21 bp, Artificial Sequence
  Decoy-Oligonucleotide
cctgtatgccgtgagctatag

SEQ ID NO: 51 - DNA, 19 bp, Artificial Sequence
  Decoy-Oligonucleotide
gccggctgaccctgcctca

SEQ ID NO: 52 - DNA, 19 bp, Artificial Sequence
  Decoy-Oligonucleotide
tcttccctagctgactgac

SEQ ID NO: 53 - DNA, 16 bp, Artificial Sequence
  Decoy-Oligonucleotide
tccctgaccgactcag

SEQ ID NO: 54 - DNA, 16 bp, Artificial Sequence
  Decoy-Oligonucleotide
tccctagctgactgac

SEQ ID NO: 55 - DNA, 27 bp, Artificial Sequence
  Decoy-Oligonucleotide
gtgcatttcccgtaaatcttgtctaca

SEQ ID NO: 56 - DNA, 22 bp, Artificial Sequence
  Primer
ctgggaactgtagtttccctag

SEQ ID NO: 57 - DNA, 22 bp, Artificial Sequence
  Primer
accctgtcattcagtgacgcac

SEQ ID NO: 58 - DNA, 22 bp, Artificial Sequence
  DNA Oligonucleotide
gctcccaccagggcatcaagct

SEQ ID NO: 59 - DNA, 16 bp, Artificial Sequence
  DNA Oligonucleotide
ttccctggccggctga

SEQ ID NO: 60 - DNA, 22 bp, Artificial Sequence
  Primer
ggatgtggctgtctgcatggac

SEQ ID NO: 61 - DNA, 22 bp, Artificial Sequence
  Primer
tggtccacgatggtgactttgg

SEQ ID NO: 62 - DNA, 24 bp, Artificial Sequence
  Primer
gaccacagtccatgccatcactgc

SEQ ID NO: 63 - DNA, 22 bp, Artificial Sequence
  Primer
atgaccttgcccacagccttgg